(19) United States
(12) Patent Application Publication
(10) Pub. No.: US 2017/0216555 A1
(43) Pub. Date: Aug. 3, 2017

(54) AUTOMATIC GENERATION OF VISUAL STIMULI

(71) Applicants: ART+COM AG, Berlin (DE); GRAFT GESELLSCHAFT VON ARCHITEKTEN MBH, Berlin (DE)

(72) Inventors: Alawi LÜTZ, Berlin (DE); Claudia SPIES, Berlin (DE); Jing HE, Berlin (DE); Jussi ÄNGESLEVÄ, Berlin (DE); Thomas WILLEMEIT, Berlin (DE)

(73) Assignees: ART+COM AG, Berlin (DE); GRAFT GESELLSCHAFT VON ARCHITEKTEN MBH, Berlin (DE)

(21) Appl. No.: 15/500,894

(22) PCT Filed: Jul. 31, 2015
(86) PCT No.: PCT/EP2015/067730

§ 371 Date: Jan. 31, 2017

(30) Foreign Application Priority Data
Aug. 1, 2014 (DE) 10 2014 215 211.9

(51) Int. Cl. A61M 21/00

(52) U.S. Cl. CPC A61M 21/00; A61M 2021/005; A61M 2205/3561; A61M 2205/502; A61M 2230/50; A61M 2230/30; A61M 2230/06; A61M 2230/42

(57) ABSTRACT

A control device (1) is disclosed for controlling a playback device (11), which is designed for playing back pictorial content on a display means (111) of the playback device (11) to generate visual stimuli (2) for a person (3). The control device comprises: an acquisition device (13), which is designed to acquire person-related data (131-1; 133-1), which are indicative of at least one symptom of the person (3), and to provide an acquisition result (13-1) depending on the person-related data; a controller (14), which is designed to determine at least one value of a playback parameter set (14-1) depending on the acquisition result; and a control unit (12), which is designed to control the playback of pictorial content (12-1), which is parameterized depending on the playback parameter set (14-1) determined with respect to value.

### BACKGROUND

The present invention relates to a control device according to the preamble of patent claim 1, a playback device according to the preamble of patent claim 18, a method for controlling a playback device according to the preamble of patent claim 19, and a corresponding computer program according to patent claim 20.

In particular, the present invention relates to an automatic generation of visual stimuli by means of a playback device for playing back pictorial content on a display means of the playback device.

It is known from DE 102 33 960 A1 or US 2009/0156886 A1 that pictorial content which is played back on a display means which is situated in the field of vision of a person, i.e., for example, videos, photos, sequences of photos, video animations, etc., results in the generation of visual stimuli, which can have an effect in particular on the state of the person, for example, on the emotional state of the person, who is confronted with the pictorial content.

### DESCRIPTION

It can be desirable to have the generation of such visual stimuli advantageously occur in an automated and deterministic manner.

The subjects of the independent patent claims are proposed according to the invention. Features of several embodiments are indicated in the dependent claims.

A first aspect is formed by a control device for controlling a playback device. The control device is designed for the playback of pictorial content on a display means of the playback device, to generate visual stimuli for a person. The control device comprises: an acquisition device, which is designed to acquire person-related data, which are indicative of at least one symptom of the person, and to provide an acquisition result depending on the person-related data, wherein the acquisition device for acquisition of the person-related data comprises: a sensor device, which is embodied for coupling to the person, to ascertain person-related data in the form of items of sensor information via the at least one symptom of the person, wherein the sensor device comprises a plurality of sensors, wherein the items of sensor information about the at least one symptom comprise values of physical measured variables, wherein each of the sensors is designed to determine a value of a specific physical measured variable; a controller, which is designed to determine at least one value of a playback parameter set depending on the acquisition result, wherein the controller is furthermore designed to in order to determine values of a number of selected physical measured variables; and the acquisition device is designed to provide the acquisition result depending on the determined values; and a control unit, which is designed to control the playback of a pictorial content, which is parameterized depending on the playback parameter set determined with regard to value.

A second aspect is formed by a method for controlling a playback device, which is designed to play back pictorial content on a display means of the playback device, in order to generate visual stimuli for a person. The method comprises: acquiring person-related data which are indicative of at least one symptom of the person, and providing an acquisition result depending on the person-related data, wherein the acquisition of the person-related data occurs via a sensor device, which is embodied for coupling to the person, to ascertain person-related data in the form of items of sensor information about the at least one symptom of the person, wherein the sensor device comprises a plurality of sensors, wherein the items of sensor information about the at least one system comprise values of physical measured variables, wherein each of the sensors is designed to determine a value of a specific physical measured variable; determining at least one value of a playback parameter set depending on the acquisition result, wherein the determination comprises a selection of a number of the plurality of sensors depending on the items of sensor information, to determine values of a number of selected physical measured variables; providing the acquisition result depending on the determined variables; and controlling the playback of a pictorial content, which is parameterized depending on the playback parameter set determined with regard to value.

### BRIEF DESCRIPTION OF THE FIGURES

The concept on which the invention is based will be explained in greater detail hereafter on the basis of the exemplary embodiments illustrated in the figures. In the figures:

FIG. 1 shows a schematic and exemplary illustration of an embodiment of a control device;

FIG. 2 shows a schematic and exemplary illustration of pictorial content which has been parameterized depending on the time of day;

FIG. 3 shows a schematic and exemplary illustration of a diagram for illustrating a parameterization of pictorial content; and

FIG. 4 shows a schematic and exemplary illustration of a flow chart to illustrate a selection of sensors of the sensor device.

### DETAILED DESCRIPTION

The control device is used for controlling a playback device, which is designed for playing back pictorial content on a display means of the playback device, to generate visual stimuli for a person.

The control device comprises an acquisition device, which is designed to acquire person-related data, which are indicative of at least one symptom of the person. The acquisition device is furthermore designed to provide an acquisition result depending on the acquired person-related data.

A controller, which receives the acquisition result and is designed to determine at least one value of a playback parameter set depending on the acquisition result, is coupled to the acquisition device, for example, connected downstream.

In addition, the control device comprises a control unit, which is designed to control the playback of a pictorial content parameterized depending on the playback parameter set, which is defined with respect to value. The control unit outputs the parameterized pictorial content on the display means.

In the similarity thereof, the controller and the control unit are accordingly designed, for example, to transform the acquisition result into the parameterized pictorial content, which can then be played back by the control unit on the display means of the playback device.

The present invention proceeds from the following findings and comprises the following basic concepts:

A person, who is to be treated, for example, because of an illness and/or an injury, i.e., for example, is treated as an inpatient in a hospital, frequently displays a certain number of undesired symptoms. Some symptoms occur more frequently than others in this case, wherein certain symptoms can influence the healing process in the person to different extents. To alleviate such symptoms or syndromes, medications are frequently taken, which often in turn result in other undesired side effects and/or symptoms, however. It can therefore be advantageous to favor non-pharmacological measures over the pharmacological measures for healing the person, or at least to use them additionally in the treatment or prevention of symptoms, in order to reduce the medication dose.

The present invention presumes, inter alia, that visual stimuli are among such non-pharmacological measures.

In the known subjects for generating such visual stimuli, however, as are described, for example, in the citations [1] to [9], it is problematic that the subjects all enable little flexibility in the generation of the visual stimuli and an adaptation of the pictorial content to different symptoms or symptom configurations (also referred to as syndromes hereafter) or to degrees of severity of symptoms are not possible. This aspect in particular is of great relevance in the intensive-inpatient context, however. This is because critically ill patients usually have a variety of symptoms of differing severity, which occur simultaneously and thus form individual syndromes. In this case, significant fluctuations of the symptom configurations can occur with specific syndromes, in particular multiple times a day.

The known subjects for generating visual stimuli are not designed to adapt the pictorial content to the fluctuations just described. Therefore, an immediate adaptation of visual contents to changed system configurations is not possible using the previously known technical solutions. This has the result that the display of the non-adapted pictorial content produces visual stimuli which not only cannot be effective for the healing process, but rather can also have negative effects.

In contrast thereto, for example, one embodiment of the control device outputs parameterized pictorial content, which considers the at least one symptom of the person, for example, a symptom complexity, a symptom fluctuation, and/or a degree of severity of presently existing symptoms in the person, so that an adaptation of the pictorial content, that is to say the parameterization of the pictorial content, to the present situation of the person can be performed more or less in real time and, for example, automatically and deterministically. The parameterization of the pictorial content by the controller is thus performed, for example, not in a random manner, but rather in a deterministic manner depending on the acquisition result, on the one hand, and with the least possible time delay, on the other hand.

For example, the control device is designed to process manually input and/or automatically acquired person-related data and to generate moving images from these data, i.e., the pictorial content for the symptom treatment and/or symptom prevention, which are to be transmitted to displaying hardware, namely the display means of the playback device, and displayed thereon.

For example, the manually input and/or automatically acquired person-related data form a test series and/or a training unit. The medical test series is brought into a logical sequence which builds on one another by the controller, for example, which both improves the performance of the test and also reduces the possibility of error in the acquisition. The training unit, which is controlled by the controller, for example, enables the person to train his cognitive capabilities automatically or manually, for example.

As explained in greater detail hereafter, the acquisition device is designed according to one embodiment to not only acquire person-related data, but rather also environmental data, for example, items of time of day and/or weather and/or position information, which the control device uses, for example, to offer the person orientation help by means of the visual stimuli. It is also possible in embodiments that the manually input and/or automatically acquired person-related data form a basis for therapeutic sessions (sittings), which can output visual biofeedback depending on the type of therapy, which will also be explained in greater detail at a later point.

In the control device, for example, a user interface assumes the role of an input interface, via which the person-related data can be input and/or automatically acquired and with which a user of the control device can take influence on the playback of the parameterized pictorial content by input of corresponding user inputs. The controller of the control device assumes the role of a type of middleware, for example, which receives all acquired data in the form of the acquisition result and possible user inputs, analyzes them, and determines the playback parameter set with respect to value depending on the analysis and provides it to the control unit. The control unit of the control device finally assumes the role of a type of renderer, which outputs the pictorial content, which is parameterized depending on the playback parameter set, on the display means of the playback device.

The display means in the viewing direction of the patients is modulated, for example, using a parametric design having different layers.

Instead of using, for example, different scenes for different symptoms or syndromes, the control device generates, for example, an assembled dynamic scene, the individual components of which can be weighted according to the existing symptoms and/or syndromes and the respective strength thereof and also further contextual data.

The control device is therefore used, for example, for controlling a display means in the form of a stimulating projection screen, which supplies adaptive cognitive excitations adapted to the state of the person in the form of the visual stimuli and is to assist the recovery of the person.

Optional components and several optional aspects of the control device will be described in greater detail hereafter:

### Playback Device Having Display Means

The display means of the playback device comprises, for example, a large-format screen on which media can be played, which can be attached above the bed, for example, on the room ceiling, and/or is designed for fastening on a wall, for example.

The display means comprises, for example, an LED grid, which can be controlled by the control device, for example, by the control unit, via a DMXKiNet protocol. The size and the resolution of the display means are variable, for example. For example, the LEDs of the display means can reproduce all colors in the RGB color space.

For example, the control device furthermore comprises a light sensor, which is designed to determine a luminosity and/or a light temperature (also referred to as a color temperature) of an ambient light, which surrounds the person, so that the control unit can adapt the brightness of the display means and/or other display parameters depending on the ambient light.

It is furthermore possible that the playback device has, in addition to the display means, a lighting device, which is designed for lighting a room. The control device can be designed to control the lighting device based on the playback parameter set, i.e., for example, depending on the luminosity and/or light temperature of the ambient light. For this purpose, the control of the lighting device is performed, for example, according to the DALI (Digital Addressable Lighting Interface) or the DMX (Digital Multiplex) protocol.

In one embodiment, the control device is designed to operate alternately in one of the following modes depending on the acquisition result:
a normal mode, in which the visual stimuli are generated depending on the acquisition result;
an emergency mode, in which the generation of the visual stimuli is stopped.

In the normal mode, the generation of the visual stimuli is performed, for example, as described above. However, if the controller recognizes, for example, that the acquisition result is indicative of a critical state of the person (such as a cardiac insufficiency), the generation of the visual stimuli is thus stopped. For example, the control device then operates the lighting device such that personnel passing the person are made aware of the critical state of the person. For example, it is conceivable that the control device operates the lighting device such that it emits a warning light or the like.

In a further embodiment, the control device is designed to control the lighting device according to a circadian rhythm. A sleep-waking rhythm of the person can be assisted in this manner.

The control device is, for example, coupled in a wired or wireless manner to the display means and optionally to the lighting device of the playback device. As a result of this coupling, the control unit can transmit data which contain the parameterized pictorial content to the display means and output them there, and/or transmit control signals to the lighting device.

### Pictorial Content and Visual Stimuli

The pictorial content relates, for example, to moving images which are parameterized by the control device automatically and in real time. The pictorial content comprises, for example, a stationary image, a sequence of stationary images, moving images, video animations, videos and/or audio contents and/or other content elements. Data which contain such pictorial content are parameterized by the control device and transmitted to the display means, to generate visual and/or audiovisual stimuli thereon. Specific examples of the pictorial content will be specified hereafter in summary:
A starry sky, which is shown slowly moving by the display means, for example, and for which the playback parameter set specifies, for example, how fast the stars move, how many stars are displayed, a density distribution, etc.
A sky background having a sun, wherein the playback parameter set determines, for example, depending on the time of day and/or the geographical position of the person, at which point the sun is to be shown, how large and bright it is, and how the color of the sky is embodied, etc.
Clouds, for which the playback parameter set specifies, for example, how rapidly they are to move, which dimensions, embodiment, and which color they are to have. The playback parameter set is determined in this case, for example, depending on present actual weather data, which have been previously ascertained by the acquisition device.
Foliage, for example, a number of leaves, wherein the density of the leaves, the scaling, and/or the movement of the leaves are specified by the playback parameter set.
A two-colored or multicolored light surface, wherein the speed and the direction of color changes on the display means are specified by the playback parameter set.
Moving spots of light, wherein the speed of the movements and a density of the moving spots of light on the display means are specified by the playback parameter set.

The parameterized pictorial content comprises, for example, passive contents and/or active contents.

The passive contents relate, for example, to a parameterized pictorial content which runs automatically and does not require an active intervention of the person. For the parameterization of the passage pictorial content, for example, clinic personnel carry out a measurement series on the person by means of the acquisition device, for example, so that the controller, for example, based on a deterministic algorithm, can determine the playback parameter set depending on the acquisition result, based on which the parameterization of the pictorial content is performed. However, according to another exemplary embodiment, the measurement series can be carried out solely by the control device, for example, i.e., without personnel.

The active contents relate, for example, to visual contents, with which the person can enter into active interaction. The person can influence, for example, the parameterization of the pictorial content by specific actions, such as movements of body parts, movement of the eyes/eyelids, etc., which are acquired by the acquisition device. In this manner, for example, therapeutic sessions with the person can be carried out by means of the control device. For example, cognitive skills can be trained, physiotherapy and/or mobilization can be assisted, and/or respiration therapy and/or ventilation withdrawal can be assisted.

In any case, according to one embodiment, the control device generates different pictorial content for different symptoms/syndromes. Because the playback parameter set is updated, for example, on the basis of continuous analyses of the person-related data, the pictorial content is adapted to the present state of the person. The control device thus enables the person to be presented with a continuous, repetition-free pictorial content.

### Person

The person is, for example, an immobile person, such as a patient, who lies in a patient room of a hospital for inpatient treatment. For example, the person is a person who is in the process of waking up from a coma or a coma-like state. In any case, the person has a physical and mental state which is time-dependent. This state of the person is ascertained by acquiring the person-related data. The person-related data are indicative, for example, of at least one of the following person-related symptoms and/or syndromes: agitation, fear, delirium, disorientation, hallucination, pain, and/or sedation. Such symptoms can occur in various combinations and to various extents. The parameterization of the pictorial content is performed depending on the symptom combination and/or depending on the strength of the individual symptoms.

### Acquisition Device, User Interface, and Person-Related Data

In general terms, the acquisition device is used to acquire all person-related data which characterize the state of the person, for example, to acquire the at least one symptom which the person has. In addition, the acquisition device can be used to acquire environmental data, which characterize the environment of the person, for example, the present time of day, the present position, the present weather, the present light conditions, etc.

The control device furthermore comprises, for the acquisition of the person-related data, for example, a user interface, which is designed to receive person-related data in the form of user inputs of a user of the control device, wherein the user inputs relate to the at least one symptom of the person.

In addition, the acquisition device can also comprise, for example, a sensor device, which is embodied for coupling to the person, to ascertain person-related data in the form of items of sensor information about the at least one symptom of the person.

In other words, the person-related data can be manually input by means of the user interface. The acquisition device can additionally or alternatively thereto, however, also acquire the person-related data by means of the sensor device.

The user interface is coupled, for example, to an input device of the control device, which is designed to produce and transmit the user inputs. In this manner, the control device can wirelessly receive the user inputs. By means of the input device, a user—for example, the person himself or the personnel taking care of the person—of the control device can input, for example, identified clinical symptoms by means of a graphic interface and/or carry out a measurement series or interview by means of the input device, which is used to ascertain a symptom configuration presently existing in the person. The user inputs can relate not only to symptoms or syndromes of the person, but rather also control instructions, which the control device is to consider during the playback of the parameterized pictorial content.

For example, the acquisition device is additionally coupled to a patient data management system (PDMS), to acquire further data about the person. By means of the coupling to the PDMS, the acquisition device can acquire further person-related data, for example, items of historical patient information, in an individual patient file which is assigned to the person.

For example, the input device already discussed is designed to carry out a standardized series of medical tests, to ascertain symptoms and/or syndromes in the person. This is performed, for example, in the context of an interview of the person.

In a further embodiment, the controller is designed to control an execution of a program on the input device depending on the user inputs and/or depending on the acquisition result. For example, the controller transmits control commands to the input device via the user interface, in order to control the execution of the program, for example, to thus determine a next question of an interview scheme or to determine a sensor to be used, which is to be operated in the scope of a measurement series.

The program which is carried out on the input device is, for example, an interview scheme (anamnesis scheme) or a diagnosis scheme, according to which the person is interviewed or diagnosed, respectively, by personnel. Responses to questions or diagnostic results are input into the input device and transmitted from the input devices as user inputs to the control device. Because the control of the program by the control device is performed depending on the user inputs and/or depending on the acquisition result, i.e., for example, depending on the items of sensor information, the interview or the diagnosis is not performed linearly, but rather situationally.

For example, the input device is a mobile terminal.

The input device transmits the data input into the input device, for example, wirelessly via WLAN or according to another wireless communication standard as user inputs to the control device. Depending on the received user inputs and/or depending on the acquisition result, the control device can also transmit commands back to the input device, so that based on these commands, a test series can be continued, if it has not yet been completed.

For example, the control device and the input device are thus designed to communicate with one another wirelessly.

For example, the input device is designed to transmit the user inputs in a predefined format to the control device.

According to one embodiment, the user interface, which is optionally part of the control device, enables a manual input of person-related data in the form of user inputs and the output of commands to the input device. In this manner, a user of the control device, for example, a physician or a nurse, and/or the person himself, can take influence on the parameterization of the pictorial content.

For the automatic acquisition of the person-related data, the acquisition device can furthermore have the above-mentioned sensor device having a number of sensors, to ascertain items of sensor information about the at least one symptom of the person. For example, the sensor device comprises one or more sensors, for example, validated measuring instruments, to ascertain symptoms such as agitation, fear, delirium, disorientation, hallucination, pain, and/or sedation.

The items of sensor information about the at least one symptom comprise, for example, values of physical measured variables. The physical measured variables are thus measurable variables which are suitable for determining one or more of the symptoms just mentioned. The physical measured variables comprise, for example, at least one of the following: a body temperature of the person, a blood pressure of the person, one or more measured variables which have been ascertained in the scope of an electroencephalograph carried out on the person, a heart rate, a respiratory frequency, etc.

If the sensor device comprises a variety of sensors, for example, each of which is designed to determine a value of a specific physical measured variable, the controller is thus designed, for example, to select a number of the variety of sensors, for example, depending on the received user inputs and/or the acquisition result, to determine values of a number of selected physical measured variables, and wherein the acquisition device is designed to provide the acquisition result depending on the determined values.

The controller is designed according to one embodiment to determine a sequence of the sensors of the sensor device to be used depending on the user inputs and/or depending on the items of sensor information. For example, the controller ascertains, based on the user inputs and/or based on the acquisition result, which sensor/which measuring instrument of the sensor device is to be used when, to ascertain further person-related data.

For example, an input of measurement results is performed via a controller app provided for this purpose, which is executed by the input device. Dependencies of the individual measurement results within a test series carried out on the person are considered and interpreted in this case. For example, pain in the case of a delirious person can only be detected by means of a specific sensor/a specific measuring instrument. On the other hand, i.e., in the case of a non-delirious person, the controller automatically selects, for example, general subjective pain measuring instruments for the acquisition of further person-related data.

According to one embodiment, the acquisition device is designed to acquire all data which relate to the person and the environment surrounding him, and to do so automatically and/or based on user inputs which are input via the user interface, wherein the acquisition device can additionally be designed to automatically select sensors/measuring instruments which are advantageous for acquiring the person-related data and/or environmental data based on previously acquired data and/or user inputs and to operate them to acquire the data. The data acquired by the acquisition device are provided by the acquisition device in bundled form in the acquisition result, for example, which is supplied to the controller.

### Light Sensor, Receivers for Weather Data/Position Data

The acquisition device can—as noted—be designed for acquiring environmental data and for providing the acquisition result also depending on the environmental data. For these purposes, the acquisition device comprises, for example, said light sensor, said receivers for weather data and/or position data, and/or said time measuring device.

The light sensor is designed for determining a luminosity and/or a light temperature of an ambient light which surrounds the person. This enables an adaptation of the playback of the parameterized pictorial content to the luminosity and/or the light temperature of the ambient light.

On the one hand, the fact is thus taken into consideration, for example, that the contrast is to be increased in the case of high luminosity, so that the person can also recognize the pictorial content well in the case of high luminosity, and on the other hand, the fact is to be taken into consideration that the display means also represents a light source during the playback of the parameterized pictorial content, which possibly has to take into consideration maximum or minimum luminosities, with which the person can be confronted. An adaptation of the light temperature by corresponding control of the display means can be performed in a similar manner.

In addition, the playback device can also comprise, in addition to the display means, said lighting device and the control device is designed, for example, to control the lighting device depending on the playback parameter set, i.e., for example, depending on the luminosity and/or the light temperature of the ambient light.

The receiver is designed to receive weather data and/or position data, which are indicative of the present and/or future weather in the environment of the person and/or are indicative of the location of the person. For example, the receiver of the acquisition device is coupled for this purpose to the Internet, to ascertain the weather data and/or the position data. The weather data and/or the position data could also be ascertained automatically by the receiver, however, for example, by appropriately embodied components such as a GPS receiver and/or a weather station.

The optionally provided time measuring device is designed to provide time data, which are indicative of the present time of day.

On the basis of the weather data and/or the position data and/or the time data, a further adaptation, i.e., a more accurate parameterization of the pictorial content to be played back to the present environment of the person is possible.

For example, the parameterized pictorial content comprises a rising sun in a clear sky, if the time data are indicative of a morning and the weather data indicate good weather. This also applies accordingly to evening hours, for which the parameterized pictorial content comprises, for example, a setting sun against dense clouds, if the weather data indicate a cloudy sky. Such examples may be continued further.

### Acquisition Result

The acquisition result provided by the acquisition device is provided, for example, in the form of a data set which comprises, for example, the—optionally processed—person-related data and optionally additionally the—optionally processed—environmental data.

The acquisition result is thus, for example, contained in a data set which describes the symptoms presently occurring in the person and the environment of the person.

Based on such a data set, which describes the present situation of the person and the environment of the person, the determination of the playback parameter set is performed by the controller.

### Controller

The controller determines, on the basis of the acquisition result and optionally on the basis of the user inputs, how the pictorial content to be played back is to be parameterized.

The controller supplies an adaptation of the pictorial content, on the one hand, to the symptoms present in the person and, on the other hand—optionally—to the environment of the person. The manner of the parameterization is established by the determined playback parameter set. This playback parameter set contains, for example, a number of playback parameters, for example, a playback content, a playback speed, a playback volume, a color, a contrast, a resolution, an area component of the display means used for the playback, and/or a playback frequency. For one of these or multiple of these playback parameters, the controller determines corresponding values, so that the control unit can be supplied the playback parameter set determined with respect to value.

Based on the determined playback parameter set, the control unit of the control device can play back the pictorial content parameterized to the display means.

The controller implements, for example, one or more algorithms, so that the conversion of the acquisition result into the playback parameter set is performed deterministically. The algorithms used can be adapted depending on more recent findings. Thus, for example, it is established in the algorithm in the case of which combination of symptoms and/or which degree of pronouncement of one or more symptoms when and which adaptation of the pictorial content is to be performed.

### Control Unit

The control unit outputs the parameterized pictorial content to the display means for the purpose of generating the visual stimuli depending on the playback parameter set. In other words: The control unit generates the parameterized pictorial content from the playback parameter set incoming thereto.

Furthermore, the control unit can operate the lighting device, as already explained above.

Further exemplary embodiments will be described hereafter. The additional features of these further embodiments can be combined with one another and also with the above-described optional features to form further exemplary embodiments, if they are not expressly described as alternative to one another.

In one embodiment of the control device, the control device furthermore comprises a storage unit coupled to the controller for storing pictorial content, wherein the controller is designed to parameterize the stored pictorial content depending on the acquisition result and/or the user inputs and to output the parameterized pictorial content by means of the control unit on the display means of the playback device to generate the visual stimuli. For example, specific files are stored in the storage unit, which contain basic types of pictorial content, for example, a variety of content elements, which can be parameterized by the controller depending on the acquisition result.

On the one hand, it is thus possible that the controller and the control unit automatically convert the acquisition result into the parameterized pictorial content independently of the previously stored pictorial content and, on the other hand, it is—alternatively or additionally thereto—possible that the control unit accesses the optionally provided storage unit for this purpose, to incorporate specific files having pictorial content during the generation of the parameterized pictorial content.

Of course, the storage unit can also be designed as part of the controller.

In a further embodiment of the control device, the sensor device is designed to ascertain the items of sensor information about the at least one symptom of the person during the playback of the parameterized pictorial content, so that actions of the person—who is subjected to the visual stimuli—influence the determination of the value of the playback parameter set by the controller. This embodiment thus enables a type of biofeedback, in which the parameterization of the pictorial content does not occur statically, but rather by incorporation of present actions and state changes of the person.

In a further embodiment of the control device, the control device furthermore has a data logger, which is designed to record the person-related data and/or the environmental data, wherein the acquisition device is designed, for example, to also produce the acquisition result depending on the recorded person-related data and/or depending on the recorded environmental data.

The data logger records, for example, the person-related data and/or environmental data during a predetermined time interval of, for example, several minutes, hours, or days. These recorded data also form, for example, the foundation for the determination of the acquisition result. The data logger is preferably designed to store the person-related data in encrypted form, so that data protection guidelines can be taken into consideration.

A further aspect is formed by a playback device for playing back pictorial content on a display means of the playback device, to generate visual stimuli for a person. The playback device comprises a control device according to the first aspect. For example, the playback device is designed for an arrangement in a patient room, for example, in a recovery room for a coma patient.

Still a further aspect is formed by the above-described method for controlling a playback device.

Still a further aspect is formed by a computer program for operating a playback device, comprising machine-readable code which, when it is executed on a control device of the playback device, is designed to cause the playback device to carry out said method.

The further aspects just described of the present invention share the advantages of the control device of the first aspect and have embodiments which correspond to the above-described embodiments of the control device, in particular as they are indicated in the dependent claims. Reference is thus made to the above statements.

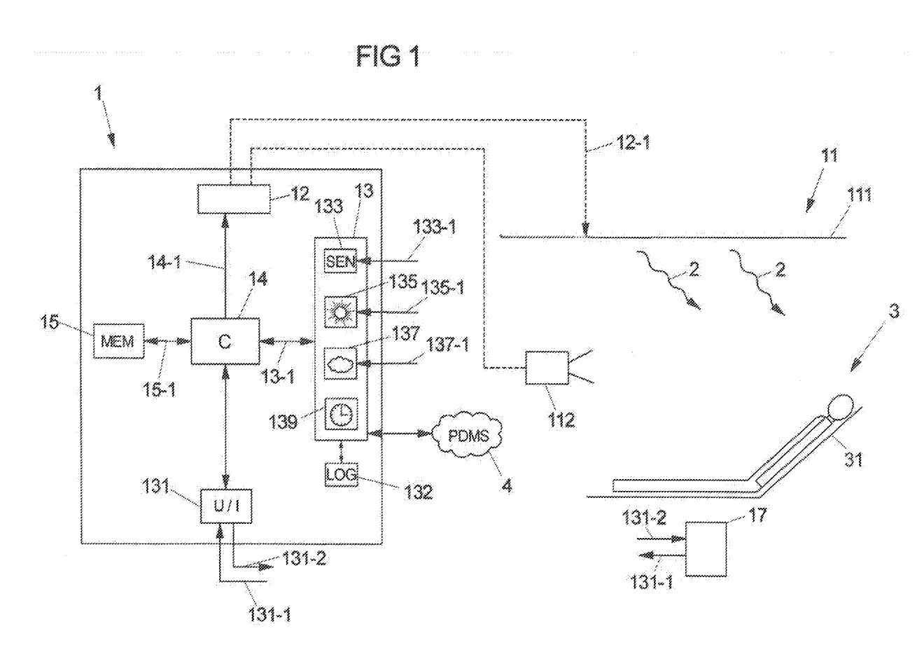

FIG. 1 shows a schematic and exemplary illustration of an embodiment of a control device 1. The control device 1 controls a playback device 11, which plays back parameterized pictorial content 12-1 on a display means 111 of the playback device 11, to generate visual stimuli 2 for a person 3. In the example shown, the person 3 is a patient 3, who lies on a couch 31.

For example, the patient 3 is to pass through a recovery process after a coma. To assist this recovery process, the patient is subjected to visual stimuli 2, the production of which is controlled by the control device 1. In the field of vision of the patient 3, the display means 111 is arranged, which is only schematically shown in FIG. 1. For example, the display means 111 is arranged on a room ceiling and/or on a room wall.

According to one embodiment, the control device 1 has an acquisition device 13 for acquiring person-related data, which are indicative of at least one symptom of the patient 3, and for acquiring environmental data, wherein the acquisition device 13 provides an acquisition result 13-1 depending on the person-related data and environmental data. This acquisition result 13-1 is supplied to a controller (C) 14, which determines a value of a playback parameter set 14-1 depending on the acquisition result 13-1. A control unit 12 is coupled to the controller 14, which receives the playback parameter set 14-1 determined with respect to value and controls the playback of the pictorial content 12-1, which is parameterized depending on the determined playback parameter set 14-1, on the display means 111.

In the example shown, the acquisition device 13 for acquiring the person-related data comprises, on the one hand, a user interface (U/I) 131, which receives user inputs 131-1 of a user of the control device 1, wherein the user inputs 131-1 relate to the at least one symptom of the patient 3. In addition, the acquisition device 13 comprises a sensor device (SEN) 133, which is designed for coupling (not shown in the figures) to the patient 3, to ascertain items of sensor information 133-1 about the at least one symptom of the patient 3.

Furthermore, an input device 17 is provided, which is designed for producing and transmitting the user inputs 131-1. Said person-related data thus comprise, on the one hand, the manually input user inputs 131-1 of a user of the control device 1 and, on the other hand, the items of information 133-1 about the at least one symptom of the person 3, which the acquisition device 13 ascertains automatically by means of the sensor device 133.

The user of the control device 1 can be, on the one hand, a person who treats the patient 3, and, on the other hand, also the patient 3 himself, however. For example, a person treating the patient 3 inputs data into the input device 17, which are relevant for the status of the patient 3. This is performed, for example, by interviewing the patient 3 according to a specific interview scheme, which is executed on the input device 17. For this purpose, the input device 17 can also be controlled wirelessly by the control device 1 by means of corresponding commands 131-2, to control the sequence of the interview of the patient 3. The input device 17 is, for example, a tablet computer or another mobile terminal. The input device 17 relays the user inputs 131-1 to the user interface 131 of the control device 1.

The sensor device 133 can have a variety of sensors/measuring instruments (not shown in FIG. 1), wherein each of the sensors is designed to determine a value of a specific physical measured variable, and wherein the controller 14 is designed to select a number of the variety of sensors, for example, depending on the received user inputs 131-1, to determine values of a number of selected physical measured variables, and wherein the acquisition device 13 is designed to provide the acquisition result 13-1 depending on determined values. Thus, for example, a variety of sensors/measuring instruments are provided, of which one or more are selected by the controller 14, to ascertain further person-related data in the form of items of sensor information 133-1 about the at least one symptom of the patient 3 by way of the selected sensors/measuring instruments.

As a result, the units 131 and 133 are used, based on manual user inputs 131-1 and based on measured values of specific physical measured variables, to ascertain specific symptoms and/or syndromes of the patient 3. Such symptoms include the following, for example: agitation, fear, delirium, disorientation, hallucination, pain, and/or sedation.

Such symptoms or symptom combinations can be present in the patient 3 and are to be influenced by means of the visual stimuli 2. The way in which the visual stimuli 2 are embodied, that is to say, how the pictorial content to be played back on the display means 111 is parameterized, is dependent on which symptoms or symptom combinations the patient 3 shows and to what extent these symptoms or symptom combinations are present. To thus influence the patient 3 positively by way of the visual stimuli 2, an exact identification and determination of the symptoms by the acquisition unit 13 is advantageous. The acquisition result 13-1 provided by the acquisition device 13 characterizes the status of the patient 3 with respect to the symptoms present therein.

As shown in FIG. 1, the acquisition device 13 additionally contains a data logger 132, which continuously records the person-related data 131-1 and 133-1 over a specific period of time, for example. The recorded person-related data can also be used as the basis for the parameterization of the pictorial content.

To ascertain further person-related data, the acquisition device 13 can furthermore be coupled to a patient data management system (PDMS) 4. Further data which describe the patient 3 can be stored on this system 4.

For the parameterization of the pictorial content 12-1, not only the person-related data 131-1 and 133-1 are relevant, but rather also environmental data 135-1 and 137-1, which characterize the immediate environment of the patient 3. For these purposes, the acquisition device 13 comprises a light sensor 135, which determines a luminosity and/or a light temperature of an ambient light 135-1, which surrounds the patient 3. Thus, for example, the contrast can be adapted depending on the luminosity and/or the light temperature. In addition, the display means 111 itself represents a not insignificant light source, wherein the patient 3 in certain cases cannot be subjected to a luminosity and/or a light temperature which exceeds or falls below a specific maximum value or a specific minimum value, respectively. As a result of the light sensor 135, such specifications can advantageously be taken into consideration, for example, also if the playback device 11 not only comprises the display means 111, but rather also a controllable lighting device 112, as has already been explained in greater detail in the general part of the description.

In addition, the acquisition device 13 comprises a receiver 137 for receiving position data and/or weather data 137-1, which are indicative of the present and/or future weather in the environment of the patient 3 or are indicative of the location of the patient 3. The receiver 137 can be coupled for these purposes, for example, to the Internet (not shown in FIG. 1) or to an intranet (also not shown), to ascertain the relevant data. The receiver 137 can also comprise corresponding means to ascertain the position data or weather data 137-1 automatically, for example, thus a GPS receiver and/or a weather station.

Finally, the acquisition device 13 comprises a time measuring device 139, which indicates the present time of day, for example.

Based on the person-related data and on the environmental data, the acquisition device 13 ascertains the acquisition result 13-1. The acquisition result 13-1 is provided, for example, in the form of a data set, which specifies, on the one hand, which symptoms or which symptom combinations the patient 3 has, to what extent they are present, and how the environment of the patient 3 is embodied.

The controller 14 receives the acquisition result 13-1 and determines, based thereon, the playback parameter set 14-1. The playback parameter set 14-1 comprises at least one playback parameter, for example, a playback content, a playback speed, a playback volume, a color, a contrast, a resolution, an area component of the display means used for the playback, and/or a playback frequency. The controller 14 thus determines in which manner which pictorial content is to be played back on the display means 111.

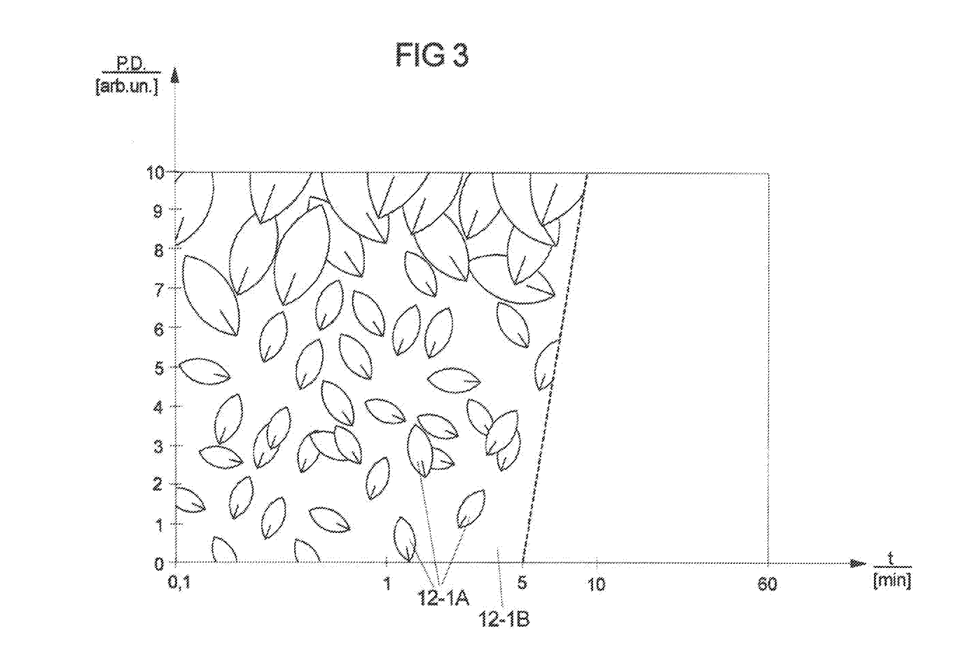

An example of a parameterization is shown in FIG. 3.

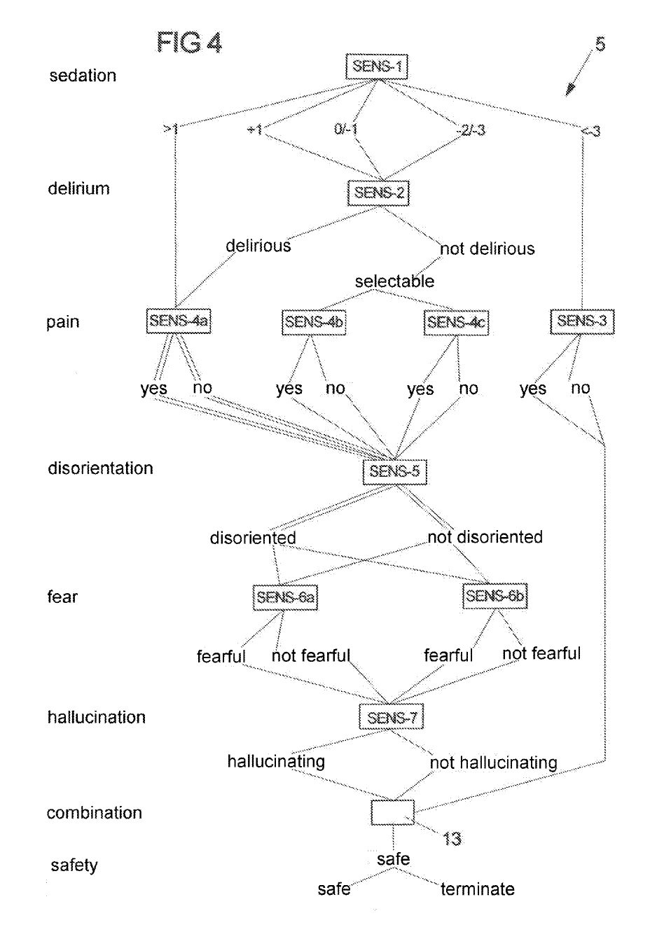

Essentially, two content elements 12-1A and 12-1B are shown therein. The first content element 12-1A is leaves and the second content element 12-1B is a background design. In the diagram shown in FIG. 4, the time in minutes is plotted on the abscissa axis and person-related data (P.D.) in an arbitrary unit (arb.un.) is plotted on the ordinate axis.

For example, the intensity of the pain sensation in the patient 3 is shown on the ordinate axis, wherein zero stands for minor pain and 10 stands for great pain. In the example shown, the controller 14 thus performs a parameterization such that with increasing pain, the size of the leaves 12-1A increases and with increasing time, the density of the leaves 12-1A shown on the background 12-1B increases. The color of the background 12-1B does not change depending on the pain intensity, but rather only depending on the time; with increasing time, the background 12-1B becomes darker.

It is to be illustrated on the basis of the example shown in FIG. 3 how an exemplary parameterization can be performed by the controller 14. The way in which the pictorial content is to be parameterized is determined by the controller 14 by the playback parameter set 14-1, which is supplied to the control unit 12. The control unit 12 outputs the parameterized pictorial content 12-1 on the display means 111.

In the example shown in FIG. 1, the control device 1 furthermore comprises a storage unit (MEM) 15, which is coupled to the controller 14. For example, files having predetermined pictorial content 15-1 are stored on the storage unit 15, which the controller 14 can access to parameterize them depending on the acquisition result 13-1. The storage unit 15 is an optional unit of the control device 1, however.

In principle, the acquisition device 13, the controller 14, and the control unit 12 are designed in their commonality to produce the parameterized pictorial content 12-1 based on the input data 131-1, 133-1, 135-1, and 137-1, and optionally based on data which are received from the patient data management system 4. Said input data are thus transformed completely automatically and deterministically into the parameterized pictorial content 12-1.

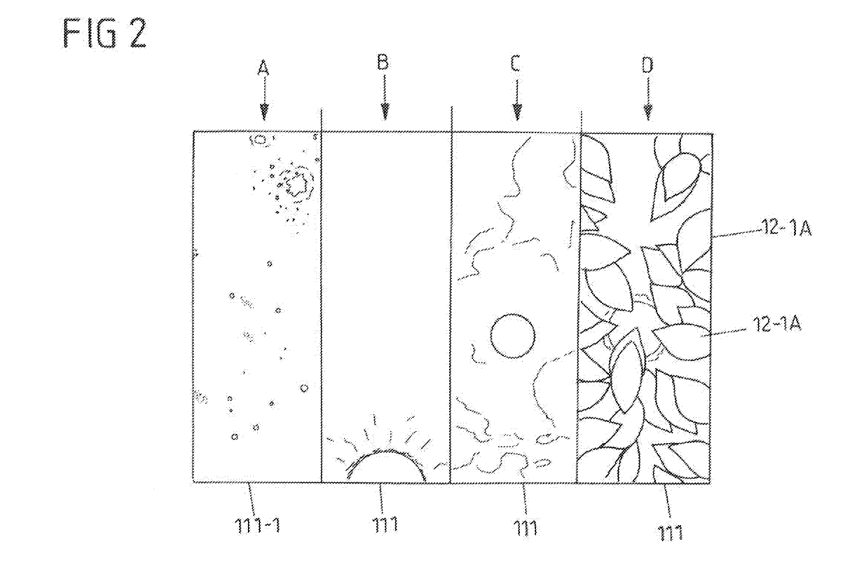

FIG. 2 shows pictorial content in schematic and exemplary form, which is played back by the display means 111 and has been parameterized depending on the time of day. The time of day is ascertained by the time measuring device 139.

In the nighttime, for example, a contact element in the form of a starry sky is displayed (variant A). Variant B shows pictorial content comprising a rising sun, which is displayed on the display means 111 in the morning, for example. Depending on the weather data 137-1, for example, a sun having a cloudy background is shown in the daytime (see variant C) and at a later time, in addition to the sun, said first content element 12-1A in the form of leaves is shown. However, FIG. 2 only shows a parameterization depending on the time of day, but not a parameterization depending on person-related data 131-1 and 133-1, wherein such a parameterization occurs in any case. In addition to the parameterization of the pictorial content depending on the person-related data 131-1 and 133-1, however, a parameterization can also be performed depending on environmental data, which has been explained with reference to the example according to FIG. 2 on the basis of environmental data in the form of time data.

Finally, FIG. 4 shows on the basis of an exemplary flow chart 5 how the controller 14 determines, depending on the user inputs 131-1 and/or depending on the items of sensor information 133-1, a sequence of the sensors to be used in the sensor measuring device 133, for example, certified measuring instruments, to obtain further person-related data in the form of further user inputs 131-1 and/or further items of sensor information 133-1, thus, for example, to ascertain the symptoms existing in the person 3:

In this exemplary embodiment, the sensor device 133 comprises the sensors SENS-1 to SENS-7. Although “sensors” are now referred to hereafter, the sensors can be, for example, certified measuring instruments or other measuring devices.

To ascertain the symptoms present in the person 3, firstly a first sensor SENS-1 is used, to determine a degree of a sedation. The controller 14 thus determines that firstly the first sensor SENS-1 has to provide corresponding items of sensor information 133-1. The first sensor SENS-1 thus acquires the value of a physical measured variable which is indicative of the degree of the sedation. If the degree of the sedation is greater than 1, for example, the controller 14 thus determines that in a next step, the sensor SENS-4a is to be used to ascertain a degree of pain of the person 3. If the degree of the sedation is thus in the range between +1 and −3, the controller 14 thus determines that in a next step, it is to be ascertained by means of the sensor SENS-2 whether the person is delirious or not. If the degree of sedation is less than −3, the controller 14 thus determines that in a next step, the sensor SENS-3 is to be used to in turn ascertain a degree of pain of the person 3.

The sensor SENS-2 of the sensor device 133 determines whether the person 3 is delirious or not. If this is the case, in a next step the sensor SENS-4a is thus selected to in turn ascertain the degree of pain of the person 3. If the person is not delirious, thus either the sensor SENS-4b or the sensor SENS-4c is selected to in turn ascertain another degree of pain of the person 3. The sensors SENS-4a, SENS-4b, SENS-4c, and SENS-3 are thus all used to ascertain whether a specific type of pain is present in the person 3 or not. The controller 14 then determines the sensor to be used depending on the results of these sensors.

For example, the controller 14 determines that in a next step it is to be ascertained by means of the sensor SENS-5 whether the person 3 is disoriented or not disoriented. Depending on the result, it is then checked by means of the sensors SENS-6a and SENS-6b whether the person 3 is in a fear state. Two sensors SENS-6a and SENS-6b which are different from one another are again provided for ascertaining the symptom.

Finally, it is checked using the sensor SENS-7 whether the person 3 is hallucinating or not hallucinating.

The collected measurement results are supplied to the acquisition device 13 in the form of the items of sensor information 133-1. For archiving purposes, the measurement results recorded during the measurement series can be secured in the data logger 132, for example.

The items of sensor information 133-1 obtained by means of the measurement series shown in FIG. 4, which have been ascertained in the illustrated sequence, for example, have direct influence on the acquisition result 13-1 and therefore on the playback parameter set 14-1, which establishes the manner of the parameterization of the pictorial content. Depending on the sequence in which the individual sensors SENS-1 to SENS-7 of the sensor device 133 are operated, the obtained items of sensor information 133-1 differ, and therefore also the pictorial content 12-1, which is played back to generate the visual stimuli 2 on the display means 111.

The control device 1 does not have to be implemented in the form of an integrated module; rather, the components, for example, the control unit 12, the controller 14, the memory 15, the acquisition unit 13 with its subunits 131, 132, 133, 135, 137, and 139 can also be arranged distributed from one another. The individual components can alternately be coupled to one another in a wired or wireless manner. In particular, for example, it is possible that the sensor device 133 comprises a variety of measuring instruments/sensors, which are arranged distributed and are coupled to the patient 3, for example, and transmit measured values to the control device 1, for example, in a wired or wireless manner.

### CITATIONS

[1] Seong-Hyun Park and Richard H Mattson. Ornamental indoor plants in hospital rooms enhanced health outcomes of patients recovering from surgery. J Altern Complement Med, 15(9):975-80, September 2009
[2] R S Ulrich. View through a window may influence recovery from surgery. Science, 224(4647):420-1, April 1984
[3] R S Ulrich, O Lunden, and Etinge J L. Effects of exposure to nature and abstract pictures on patients recovery from heart surgery. Psychophysiology, pages S1-7, 1993
[4] Quinn M. Biggs, Kimberly S. Kelly, and J. David Toney. The effects of deep diaphragmatic breathing and focused attention on dental anxiety in a private practice setting. J Dent Hyg, 77(2):105-13, 2003
[5] R. Colombo, A. Corona, F. Praga, C. Minari, C. Giannotti, A. Castelli, and F. Raimondi. A reorientation strategy for reducing delirium in the critically ill. results of an interventional study. Minerva Anestesiol, 78(9):1026-33, 9 2012
[6] S Kühn, T Gleich, R C Lorenz, U Lindenberger, and J Gallinat. Playing super mario induces structural brain plasticity: gray matter changes resulting from training with a commercial video game. Mol Psychiatry, October 2013
[7] J A Anguera, J Boccanfuso, J L Rintoul, O Al-Hashimi, F Faraji, J Janowich, E Kong, Y Larraburo, C Rolle, E Johnston, and A Gazzaley. Video game training enhances cognitive control in older adults. Nature, 501(7465):97-101, September 2013
[8] Aimee Spector, Lene Thorgrimsen, Bob Woods, Lindsay Royan, Steve Davies, Margaret Butterworth, and Martin Orrell. Efficacy of an evidence-based cognitive stimulation therapy programme for people with dementia: randomised controlled trial. Br J Psychiatry, 183:248-54, 9 2003
[9] Chia-Min M. Cheng, Ming-Jang J. Chiu, Jyh-Horng H. Wang, Hwa-Chang C. Liu, Yea-Ing Lotus I. Shyu, Guan-Hua H. Huang, and Cheryl Chia-Hui Chen. Cognitive stimulation during hospitalization improves global cognition of older taiwanese undergoing elective total knee and hip replacement surgery. J Adv Nurs, 68(6):1322-9, 6 2012

1. A control device (1) for controlling a playback device (11), which is designed to play back pictorial content on a display means (111) of the playback device (11) to generate visual stimuli (2) for a person (3), wherein the control device (1) comprises:
an acquisition device (13), which is designed to acquire person-related data (131-1; 133-1), which are indicative of at least one symptom of the person (3), and to provide an acquisition result (13-1) depending on the person-related data, wherein the acquisition device (13) for acquiring the person-related data (131-1; 133-1) has:
a sensor device (133), which is embodied for a coupling to the person (3), to ascertain person-related data in the form of items of sensor information (133-1) about the at least one symptom of the person (3), wherein the sensor device (133) has a variety of sensors (SENS), wherein the items of sensor information (133-1) about the at least one symptom comprise values of physical measured variables, wherein each of the sensors (SENS) is designed to determine a value of a specific physical measured variable;
a controller (14), which is designed to determine at least one value of a playback parameter set (14-1) depending on the acquisition result (13-1), wherein
the controller (14) is furthermore designed to select a number of the variety of sensors (SENS) depending on the items of sensor information (133-1), to determine values of a number of selected physical measured variables; and
the acquisition device (13) is designed to provide the acquisition result (13-1) depending on the determined values; and
a control unit (12), which is designed to control the playback of a parameterized pictorial content (12-1) depending on the playback parameter set (14-1) determined with respect to value.

2. The control device (1) as claimed in claim 1, wherein the acquisition device (13) for acquiring the person-related data (131-1; 133-1) has:
a user interface (131), which is designed to receive person-related data in the form of user inputs (131-1) of a user of the control device (1), wherein the user inputs (131-1) relate to the at least one symptom (3) of the person.

3. The control device (1) as claimed in claim 2, wherein the controller (14) is designed to select a number of the variety of sensors (SENS) depending on the received user inputs (131-1).

4. The control device (1) as claimed in any one of the preceding claims, wherein the controller (14) is designed to determine a sequence of the sensors of the sensor device (133) to be used depending on the user inputs (131-1) and/or depending on the items of sensor information (133-1).

5. The control device (1) as claimed in any one of the preceding claims, wherein the acquisition device (13) is furthermore designed to acquire environmental data (135-1, 137-1) and to provide the acquisition result (13-1) depending on the environmental data, wherein the acquisition device (13) has, for acquiring the environmental data (135-1, 137-1):
a light sensor (135), which is designed to determine a luminosity and/or a light temperature of an ambient light (135-1), which surrounds the person (3); and/or
a receiver (137), which is designed to receive weather data and/or position data (137-1), which are indicative for the present and/or future weather in the environment of the person (3) and/or are indicative of the location of the person (3).

6. The control device (1) as claimed in any one of the preceding claims, wherein the playback parameter set (14-1) has at least one playback parameter, for example:
a playback content;
a playback speed;
a color;
a contrast;
a resolution;
an area component of the display means (111) used for the playback; and/or
a playback frequency.

7. The control device (1) as claimed in any one of the preceding claims, wherein the control device (1) furthermore has a storage unit (15) coupled to the controller (14) for storing pictorial content, wherein the controller (14) is designed to parameterize the stored pictorial content (15-1) depending on the acquisition result (13-1) and/or depending on the user inputs (131-1).

8. The control device (1) as claimed in any one of the preceding claims, wherein the sensor device (133) is designed to ascertain the items of sensor information (133-1) about the at least one symptom of the person (3) during the playback of the parameterized pictorial content (12-1), so that the present at least one symptom of the person (3) influences the determination of the value of the playback parameter set (14-1) and therefore the parameterization of the presently played back tutorial content (12-1).

9. The control device (1) as claimed in any one of the preceding claims, wherein the person-related data are indicative of at least one of the following person-related symptoms:
agitation;
fear;
delirium;
disorientation;
hallucination;
pain; and/or
sedation.

10. The control device (1) as claimed in any one of the preceding claims, wherein the control device (1) furthermore has an input device (17), which is designed for producing and transmitting the user inputs (131-1) to the control device (1).

11. The control device (1) as claimed in claim 10, wherein the controller (14) is designed to control an execution of a program on the input device (17) depending on the user inputs (131-1) and/or depending on the acquisition result (13-1).

12. The control device (1) as claimed in any one of the preceding claims, wherein the display means (111) is designed in the form of a projection screen and is embodied for an arrangement on a room wall (51) and/or on a room ceiling (52).

13. The control device (1) as claimed in any one of the preceding claims, wherein the control device (1) furthermore has a data logger (132), which is designed to record the person-related data (131-1, 133-1) and/or the environmental data (135-1, 137-1), wherein the acquisition device (13) is designed to produce the acquisition result (13-1) in addition depending on the recorded person-related data and/or the recorded environmental data.

14. The control device (1) as claimed in any one of the preceding claims, wherein the pictorial content comprises parametrically generated real-time moving images.

15. The control device (1) as claimed in any one of the preceding claims, wherein the controller (14) and the control unit (12) are designed in their commonality to transform the acquisition result (13-1) and optionally the user inputs (131-1) into the pictorial content (12-1) to be played back.

16. The control device (1) as claimed in any one of the preceding claims, wherein the playback device (11) has a lighting device (112) for illuminating the environment of the person (3), wherein the controller (14) and the control unit (12) are designed to control the lighting device depending on the acquisition result (13-1) and/or the user inputs (131-1).

17. The control device (1) as claimed in any one of the preceding claims, wherein the control device (1) is designed to operate alternately in one of the following modes depending on the acquisition result (13-1):
a normal mode, in which the visual stimuli (2) are generated depending on the acquisition result (13-1);
an emergency mode, in which the generation of the visual stimuli (2) is stopped.

18. A playback device (1, 11) for playing back parameterized pictorial content (12-1) on a display means (111) of the playback device (11), to generate visual stimuli (2) for a person (3), comprising a control device (1) as claimed in any one of the preceding claims.

19. A method for controlling a playback device (11), which is designed to play back pictorial content on a display means (111) of the playback device (11) to generate visual stimuli (2) for a person (3), the method (1) comprising:
acquiring person-related data (131-1; 133-1), which are indicative of at least one symptom of the person (3), and providing an acquisition result (13-1) depending on the person-related data, wherein the acquisition of the person-related data is performed via a sensor device (133), which is designed for coupling to the person (3) to ascertain person-related data in the form of items of sensor information (133-1) about the at least one symptom of the person (3), wherein the sensor device (133) has a variety of sensors (SENS), wherein the items of sensor information (133-1) about the at least one symptom comprise values of physical measured variables, wherein each of the sensors (SENS) is designed to determine a value of a specific physical measured variable;
determining at least one value of a playback parameter set (14-1) depending on the acquisition result (13-1), wherein the determination comprises a selection of a number of the variety of sensors (SENS) depending on the items of sensor information (133-1), to determine values of a number of selected physical measured variables;
providing the acquisition result (13-1) depending on the determined values; and
controlling the playback of pictorial content (12-1), which is parameterized depending on the playback parameter set (141) determined with respect to value.

20. A computer program for operating a playback device, having machine-readable code which, when it is executed on a control device of the playback device, is designed to cause the circuit arrangement to carry out the method as claimed in claim 19.
